(19) United States
(12) Patent Application Publication
(10) Pub. No.: US 2015/0225783 A1
(43) Pub. Date: Aug. 13, 2015

(54) NONDESTRUCTIVE COLLECTION OF EVIDENCE

(71) Applicant: LOCKHEED MARTIN CORPORATION, Bethesda, MD (US)

(72) Inventors: John Cleon Mears, Gaithersburg, MD (US); Jeffrey A. Stuart, Cherry Hill, NJ (US)

(73) Assignee: LOCKHEED MARTIN CORPORATION, Bethesda, MD (US)

(21) Appl. No.: 14/618,164

(22) Filed: Feb. 10, 2015

Related U.S. Application Data
(60) Provisional application No. 61/937,894, filed on Feb. 10, 2014.

(51) Int. Cl. C12Q 1/68; H04N 5/225

(52) U.S. Cl. CPC C12Q 1/6869; H04N 5/2256

(57) ABSTRACT

A system and method of identifying a print includes an image-capturing and lighting optical system configured to maximize specular reflection of light reflected from a print and to minimize diffused reflection of light reflected from a background surface of the print via adjustment of at least one of a frequency and a reflection angle of the light emitted upon a sample of the print. The system and method also include an IC having one or more FETs with a nanostructure configured to detect a plurality of analytes from the print. The system and method also include a nucleic acid analyzer configured to process the print and to determine a DNA content of the print. There is no contact made with the print, while being subjected to processing by the image-capturing and lighting optical system and the IC.

### CROSS-REFERENCE TO RELATED APPLICATION

This application claims the benefit of U.S. Provisional Application No. 61/937,894, filed on Feb. 10, 2014, the disclosure of which is incorporated in its entirety by reference herein.

### BACKGROUND

Touch deoxyribonucleic acid (DNA) is a forensic method for analyzing DNA left at the scene of a crime or elsewhere. Touch DNA requires very small samples, for example from the skin cells left on an object after it has been touched or casually handled. Touch DNA analysis only requires about seven or eight cells from the outermost layer of human skin.

Techniques for collecting forensic evidence include capturing fingerprints at the crime scene or elsewhere. Fingerprints are typically dusted and lifted with sticky tape. Unfortunately, this can change the scene by destroying or rendering other potential evidence unusable. In addition, false positive results occur frequently due to contamination from fingerprint brushes used by crime scene investigators, which can transfer trace amounts of skin cells from one surface to another.

Fingerprints on portable objects are usually taken to a lab for processing, and the processing method depends on the object or surface on which the fingerprints reside. One method includes subjecting the fingerprints to cyanoacrylate fuming. In another method, paper can be treated with ninhydrin dye. However, these methods can adulterate or destroy any additional forensic value of the evidence. In addition, it can take several days to complete the processing.

### SUMMARY

Aspects of the disclosure include methods and systems for nondestructive collection and identification of evidence by latent imaging and analyte-based sensing of prints, such as fingerprints or palm prints. The DNA content of the prints is subsequently obtained.

Embodiments include a method of capturing a print, such as a fingerprint or a palm print. A latent print is illuminated on a foundation with a light. At least one of a frequency and an angle of reflection of the light is adjusted to provide maximum specular reflection of the light from the latent print and minimum diffused reflection of the light from the latent print. A resulting image of the latent print in contrast to the foundation is captured.

Embodiments include a method of identifying a print, such as a fingerprint or a palm print. An image of a sample of a latent print is located and captured on a foundation, via an adjusted frequency or an adjusted reflection angle of lighting. One or more analytes on the sample are determined, via an integrated circuit (IC) configured with one or more Field Effect Transistors (FETs) for analyte detection. A DNA content of the latent print is analyzed, via a nucleic acid analyzer subsequent to the locating, the capturing, and the determining. No contact is made with the print during the locating, the capturing, and the determining steps.

Embodiments include a system of identifying a print, which includes an image-capturing and lighting optical system configured to maximize specular reflection of light reflected from a print and to minimize diffused reflection of light reflected from a background surface of the print via adjustment of at least one of a frequency and a reflection angle of the light emitted upon a sample of the print. The system also includes an IC having one or more FETs with a nanostructure configured to detect a plurality of analytes from the print. The system also includes a nucleic acid analyzer configured to process the print and to determine a DNA content of the print. There is no contact made with the print, while being subjected to processing by the image-capturing and lighting optical system and the IC.

### BRIEF DESCRIPTION OF THE DRAWINGS

Various exemplary embodiments will be described in detail with reference to the following figures, wherein:

FIGS. 1A-1B are overviews of a nondestructive collection system according to some embodiments;

FIGS. 2-3 are illustrations of a print imaging system according to some embodiments;

FIGS. 4-5 are illustrations of a nanostructure-based electronic sensor according to some embodiments;

FIGS. 6A-6B are exemplary algorithms for training and assessing an identification model according to some embodiments;

FIG. 7 is a block diagram of an exemplary nucleic acid analyzer according to an embodiment;

FIGS. 8A-8B are illustrations of a microfluidic cartridge having a plurality of exemplary sample acceptors according to an embodiment;

FIG. 9 is a flowchart of an exemplary method of capturing a print according to an embodiment; and

FIG. 10 is a flowchart of an exemplary method of identifying a print according to an embodiment.

### DETAILED DESCRIPTION

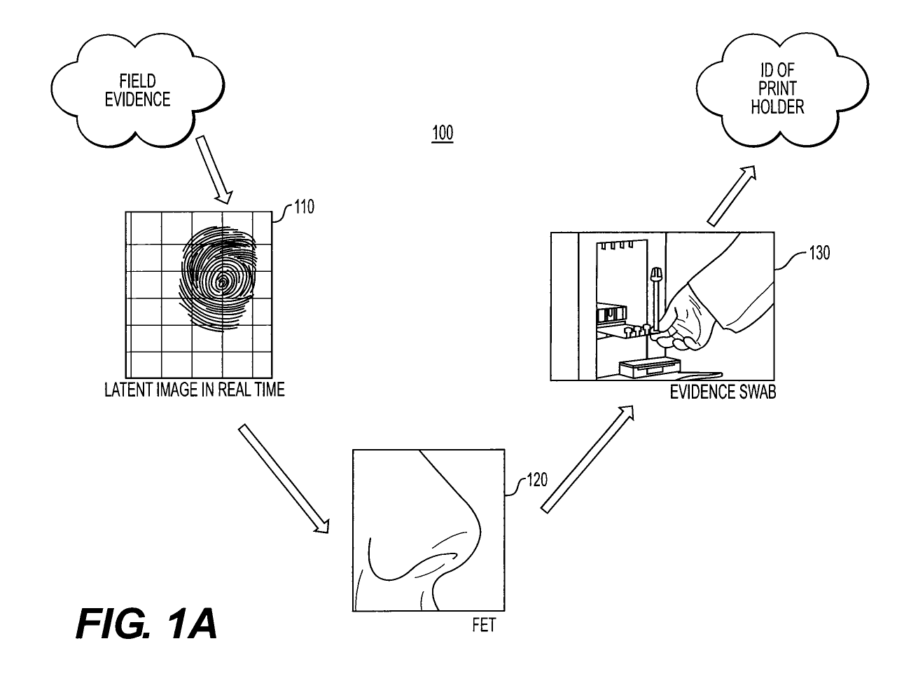

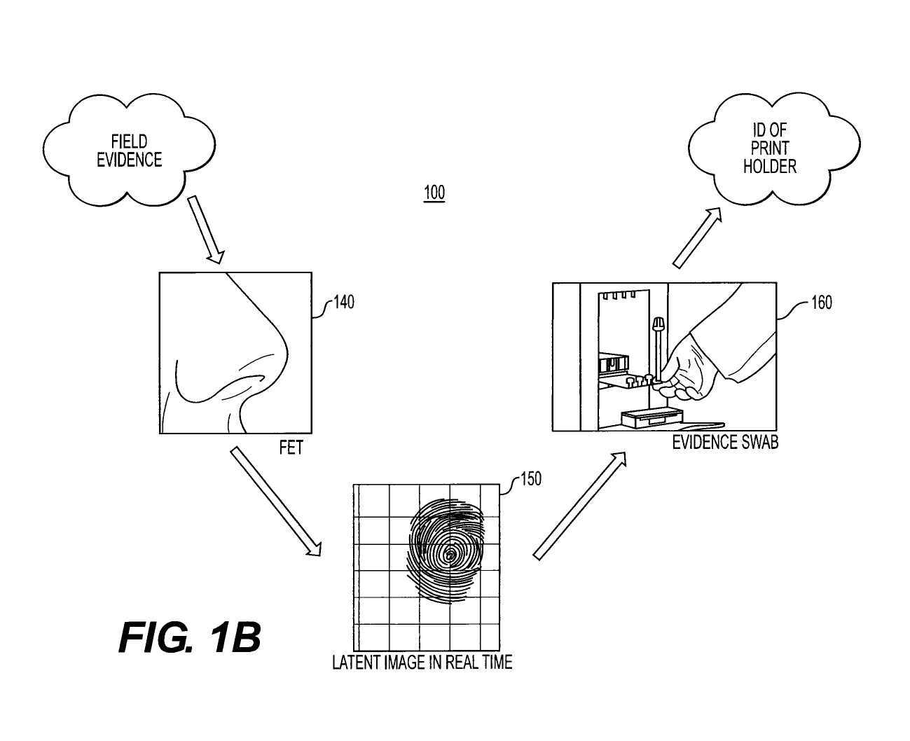

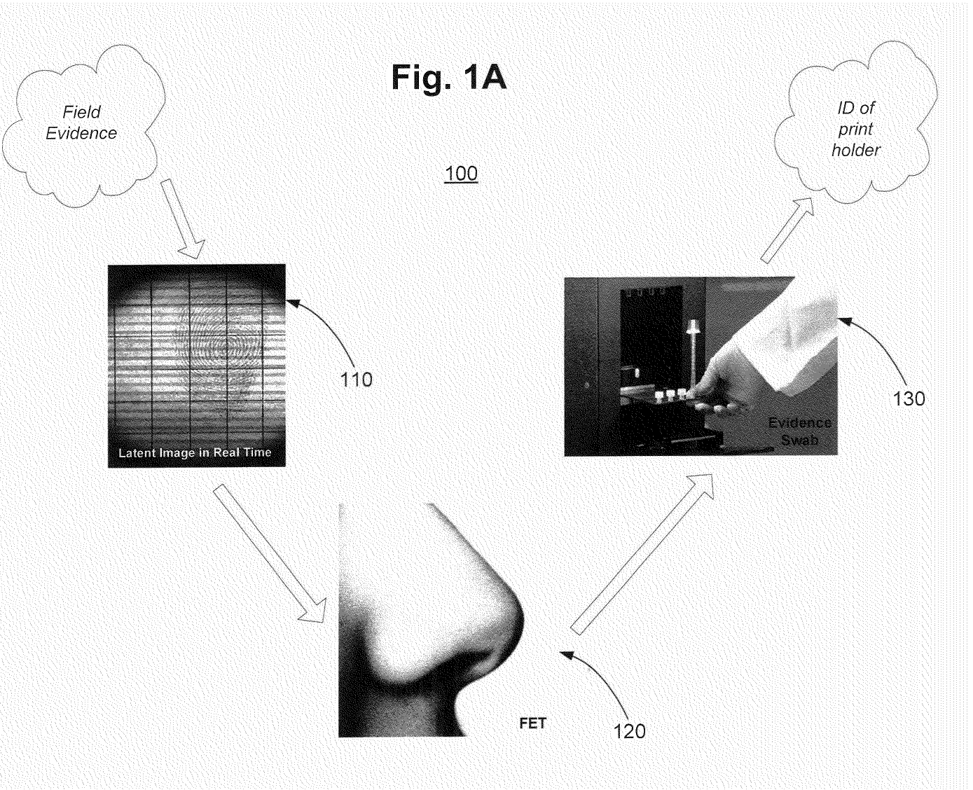

FIG. 1A is an overview of an exemplary nondestructive collection system 100 for collecting and processing field evidence, such as human palmar or plantar friction ridge prints from a crime scene. A first processing station 110 includes a system for capturing a latent image of one or more prints, such as fingerprints, footprints, toe prints or palm prints. A latent print is a print impression or residue left on a solid surface following surface contact, and is caused by physical depressions in the material due to the friction ridges or the deposition of perspiration, skin oil, or other chemicals or compounds that may reside on the ridges of an individual's skin on the finger, palm, foot or toe which comes into contact with the solid surface. The contact leaves residue and/or friction ridge depressions behind, making an impression on the solid surface. The print impression can include substances, such as water, salt, blood, amino acids, oils, grime, drugs, explosives, or dirt that may be present on a surface of the finger or palm.

Embodiments for obtaining a non-contact latent image of the prints include a light source positioned relative to a camera to utilize a specular reflection, i.e., glare from an irradiated sample surface. When arranged so that an angle of incidence from the light source to the solid surface is approximately equal to the angle of reflection from the solid surface to an image detector, the specular reflection can be maximized. With such an arrangement, a minimal amount of diffuse reflection is captured, as diffuse reflected light may lower the quality of the print image. Accordingly, when properly aligned, the light source and image detector act as a filter to discriminate highly against diffuse reflections from the solid surface by providing a geometric filter that essentially only accepts specular reflections.

In order to detect prints on a wide variety of different surfaces, such as tools, guns, phones, and phone cases, multiple different illumination wavelength bands or ranges of wavelength bands are desired. Each wavelength band can provide a different kind of light, such as white light, narrowband light, ultra-violet (UV) light, infrared (IR) light, or other specific wavelengths, wavelength ranges, or wavelength combinations of electro-optical radiation. Variation in wavelengths or wavelength ranges can be realized with one or more very broad spectrum light sources and a configurable filtering adjustment. Light sources and filtering adjustments include adjustable filters, multiple filters that can be activated or de-activated, refraction or reflection techniques that separate out only particular wavelengths or combinations thereof, or multiple individual light sources configured to produce one or more of the desired wavelengths or wavelength ranges. In some embodiments, multiple illumination wavelengths or wavelength ranges can be used, wherein light from each wavelength range may scatter off an interrogated sample surface differently. The wavelength ranges can be used one at a time with each range producing a different effect on the latent print, or multiple wavelength ranges can be combined for simultaneous illumination.

A second processing station 120 includes an integrated circuit (IC) containing one or more field-effect transistors (FETs) incorporating nanostructures for detection of volatile analytes present in the print. FETs can be chemically-based FETs for chemical detection (ChemFETs) or biologically-based FETs for detection of biologically active molecules (BioFETs). An example of an analyte is an odorant. However, other non-odorant analytes are contemplated by embodiments described herein.

This is also a non-contact system for obtaining additional information from prints or other evidence. The nanostructure-based FETs of an IC may contain one or more nanotubes, such as carbon nanotubes, which have been wrapped or engulfed with molecular agents or functionalization agents that mediate interactions between the nanostructured element of the FET with the surrounding medium. The functionalized nanostructures comprise the active medium of the FET gate. Functionalization agents (or analyte receptors) can be of biological or chemical origin, such as a strand of DNA (single or double stranded) or a protein medium from a specific analyte receptor protein. Other classes of functionalization agents for nanostructure-based FETs include, but are not limited to RNA aptamers, peptides, proteins, enzymes, polymer formulations, and other chemical coatings of the nanostructured signal-transduction surface. The functionalized nanotube comprises the active element of the gate, and is electrically connected between an electrode source and an electrode drain of the FET. When the FET is in the vicinity of a gas or liquid that interacts with the functionalized nanostructures, the FET will be activated, and thereby transmit a signal. For example, the presence of a protein may change the conductance of the nanotube, and result in a detectable change between electrodes of the FET. As a result, an analyte, such as an odorant can be detected by binding the analyte to the analyte receptor agent of the FET.

The IC can include multiple FETs, each FET being designed with a different analyte receptor agent. In an embodiment, each IC can contain a particular grouping of FETs, ChemFETs, and/or BioFETs by category. As an example for illustrative purposes only, one or more ICs can be designed to detect explosives and one or more other ICs can be designed to detect drugs. Accordingly, a bio-sensor can be an end product that contains multiple ICs.

A third processing station 130 includes a system for identifying the DNA content of the print swab, the results of which can be compared to identifying information stored in one or more databases. A biological sample, such as a print swab is contained within a microfluidic cartridge, which is inserted into a nucleic acid analyzer system. Nucleic acids are extracted from the print swab by the nucleic acid analyzer system. The extracted nucleic acids are amplified and separated for detection and analysis of the resulting DNA fragments.

Since the integrity of the print swab will be altered during the third processing station 130, this station for identifying the DNA content needs to be the last processing station. As a result, a maximum number of skin cells are provided to the third processing station 130 since the first and second processing stations leave the print swab undisturbed.

FIG. 1B is an overview of the nondestructive collection system 100 for collecting and processing field evidence, in which a first processing station 140 includes an IC containing one or more nanostructure-based FETs. As above, this IC provides a non-contact procedure of detecting various odorants and other molecules (e.g., residues from explosives, narcotics, and other illicit contraband) that may be present on field evidence, such as fingerprints or palm prints. A second processing station 150 includes a system for capturing a latent image of one or more prints. As noted above, the first processing station 140 and the second processing station 150 include systems for retrieving information from field evidence such as prints, without disturbing or contacting the field evidence.

A third processing station 160 includes a system for identifying the DNA content of the print swab. A print swab will likely have a low number of cells for testing. However, as in the previous embodiment, the number and the quality of original cells present on the print swab will be maximized for testing at the third processing station 160 since the first processing station 140 and the second processing station 150 do not disturb or contact the print swab.

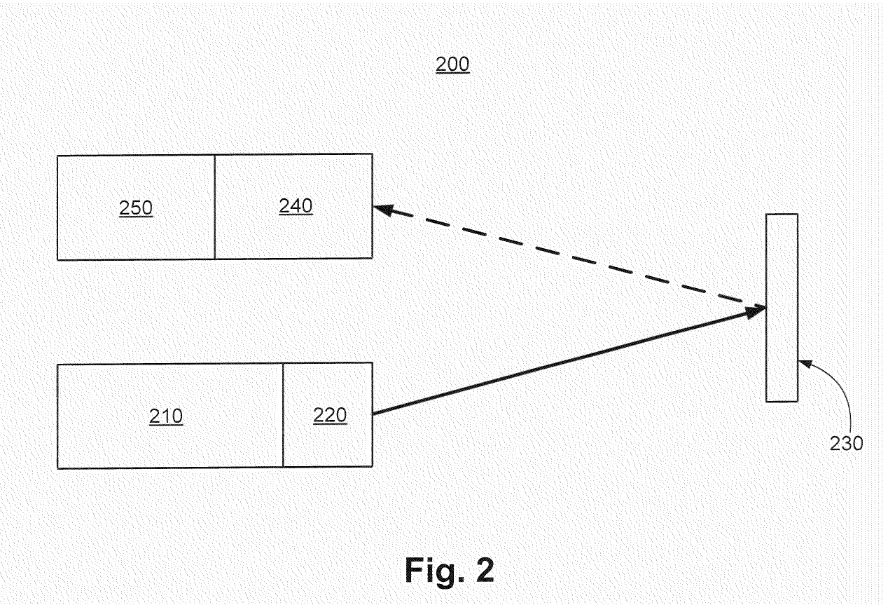

FIG. 2 illustrates an embodiment of a print imaging system 200 in a first processing station. The print imaging system 200 includes a light source 210, which can include a filter 220. Embodiments of the light source 210 can include narrow band or broad spectrum light sources in visible, IR, or UV spectra or combinations thereof. Embodiments of the filter 220 can include band-pass filters, notch filters, spectrometers, prisms, waveband-specific mirrors, filter coatings, and other devices, materials, and techniques for filtering electro-optical radiation produced in one or more specific illumination wavelength ranges. The resulting illumination can be a collimated, narrow beam directed at a surface of a sample 230. The illumination can be collimated and narrowed in order to provide increased specular reflection from the surface of the sample 230 to a detector 240.

Specular reflection, i.e., glare, can be pronounced in situations where an angle of incidence and an angle of reflection are nearly the same. Some variations can include aligning the light source 210 and the detector 240 at a critical alignment angle to facilitate the creation and capture of specular reflection from the illuminated surface of the sample 230. The critical alignment angle is one where the angle of incidence and the angle of reflection are nearly equal relative to the illuminated surface of the sample 230. This is the angle at which specular reflection is most pronounced.

By maintaining a critical alignment between the light source 210 and the detector 240, the light source 210 and detector 240 can be configured to behave as a filter that discriminates against diffuse reflections and essentially only accepts specular reflection as input into the detector 240. In some embodiments, the detector settings can be further configured to create a geometric filtering effect that causes over 90% of the photons processed by a camera to be from glare. Such a configuration can be realized by setting a numerical aperture (NA) of a lens or other optical system coupled to the detector 240 to be zero. Such a configuration can also be realized by setting the NAs of the optical system and the light source 210 to be substantially equal and opposite.

In some embodiments, the detector 240 can be coupled with or be part of an image processor 250. For example, a charge-coupled device (CCD) or a complementary metal-oxide-semiconductor (CMOS) camera device with image detection and image processing capabilities can be used. The image processor 250 can be configured to provide illumination that approaches a saturation threshold of the detector 240. However, the light source 210 should not reach the saturation threshold of the detector 240 in order to avoid loss of contrast or other loss of image data due to detector saturation.

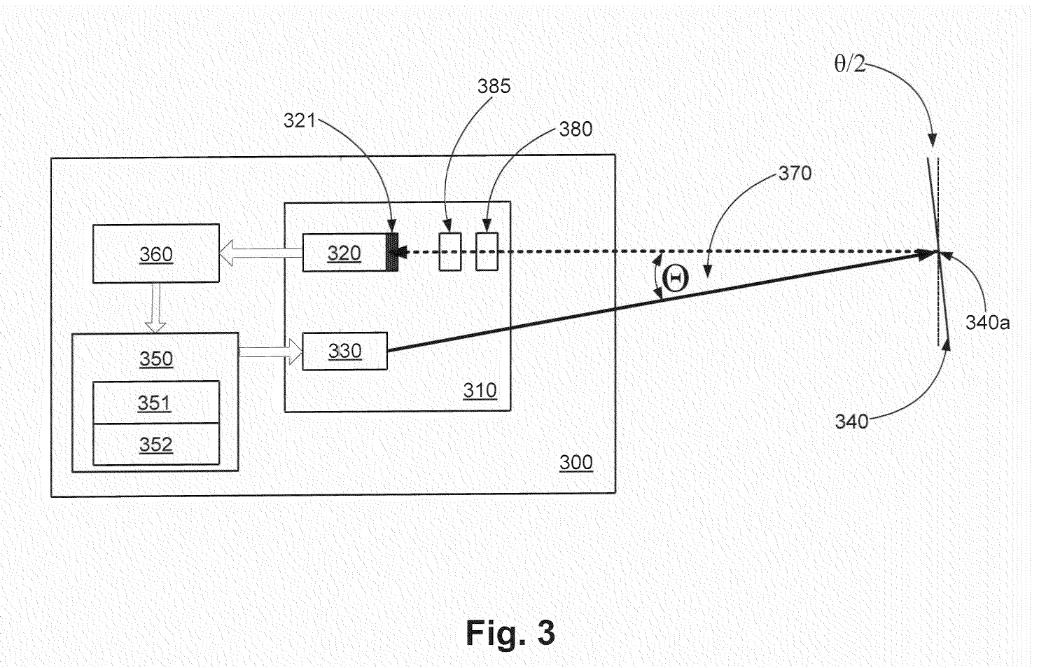

FIG. 3 is a more detailed illustration of a print imaging system 300. An optical sensor system 310 includes an image detection device 320, such as a camera or a focal plane array. In some embodiments, the image detection device 320 includes or is coupled with a lens 321 to focus incoming electro-optical radiation. In some embodiments, the image detection device 320 is a camera that is positioned at a critical alignment angle 370 in conjunction with light source 330. This provides collection of glare photons and rejection of diffusely reflected photons by configuring the NAs of the camera 320 and the light source 330 to be of equal and opposite value.

In an embodiment, the light source 330 is included in the optical sensor 310, wherein the light source 330 and the optical sensor 310 are arranged in a single housing. A similar alignment or mounting arrangement that establishes or maintains a critical alignment angle 370 between the detector 320 and the light source 330 is also contemplated by embodiments described herein. In other embodiments, the light source 330 may be physically separate from the optical sensor 310 and can be controlled or configured to provide specific illumination based on imaging parameters or requirements. Specific illuminations can include varying degrees of collimation and angle-of-incidence from the light source 330 relative to an area of interest 340a on a surface of a sample 340 that is to be imaged. The angle between the surface of the sample 340 and a vertical plane perpendicular to the reflected light is equal to one-half of the critical alignment angle 370, i.e., ½ θ.

The print imaging system 300 can also include or be connected to a computer or processor 350 to process the image data acquired by the image detection device 320. The processor 350 includes a memory 351 for storing data and a controller 352 for controlling some or all of the optical sensor components. In some embodiments, the light source 330 can be controlled to provide a uniformly extended, collimated beam onto the surface of the sample 340. In some embodiments, the image detection device 320 can be coupled to the processor 350 via a frame grabber 360, for example, an electronic device that captures individual, digital still frames from an analog video signal or a digital video stream. Other embodiments include a camera with an integrated or built-in frame grabber 360.

A latent print image can be matched against a local database (with respect to a portable computer) of suspect prints, or it can serve as a conduit to a state or local automated fingerprint identification system (AFIS) or a national database such as the FBI's Next Generation Identification (NGI) system. This facilitates near real-time feedback to the collection point on possible “owners” of the latent prints collected. The result can be displayed on a user interface of a portable computing device.

The light source 330 can be dynamically adjusted to maintain a critical alignment angle 370, as shown with respect to the image detection device 320. In some embodiments, the adjustment can be achieved with movable mirrors, refractive devices, prisms, or other combinations. In one embodiment, both the light source 330 and the image detection device 320 are secured to a fixture to maintain a critical alignment angle, even when the entire system 300 is moved.

Embodiments of the print imaging system 300 can include at least one detection filter, such as a Fourier filter 380 or a notch filter 385. Variations of the notch filter 385 can include using a laser for critical alignment purposes or as a diffuse scatter light source. Variations of the Fourier filter 380 can be used to match print features as well as suppress background features, such as grains or surface irregularities in detection on a paper or cardboard sample surface. Some embodiments include multiple detection filters, whereas other embodiments have no detection filters or have detection filters integrated into the image detection device 320.

A second processing station includes utilizing an electronic device capable of analyte detection through appropriately functionalized ChemFETs and/or BioFETs. In the human olfactory system, a specific odorant binds to an olfactory receptor protein which triggers signal transduction in a cell. Olfactory receptors within the cell membranes of olfactory receptor neurons are responsible for the detection of odorant molecules. When the odorant binds to the olfactory receptors, the receptors are activated. The activated olfactory receptors produce a nerve impulse which is transmitted to the brain. These olfactory receptors are members of the class A rhodopsin-like family of G protein-coupled receptors (GPCRs).

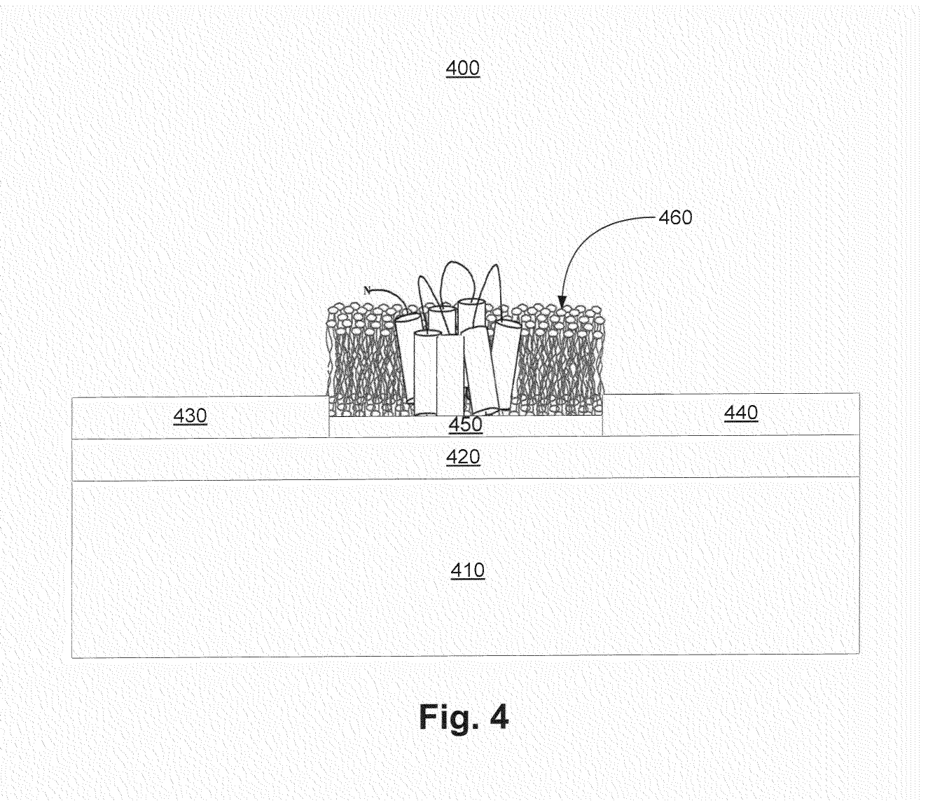

FIG. 4 illustrates an exemplary analyte-detection based electronic sensor device 400, such as an olfactory-based electronic device. A wide range of olfactory-based electronic devices, also known as “e-noses” with varying sensor types and applications are available and can be used with embodiments described herein. A substrate 410 forms the base of the analyte-detection based electronic device 400. One embodiment of the substrate 410 is a silicon substrate. However, other substrates used in electronic devices are contemplated by embodiments described herein. An oxide layer 420 resides on the substrate 410. For a silicon substrate 410, the oxide layer 420 could include silicon dioxide. A drain 430 resides on the oxide layer 420 to one side of the substrate 410 and a source 440 resides on the oxide layer 420 to another side of the substrate 410. As shown, there can be a gap residing between the source 440 and drain 430.

A nanostructure layer 450 can be arranged on the oxide layer 420 and the gate within the gap between the source 440 and the drain 430. The nanostructure layer 450 contacts the source 440 and the drain 430. Embodiments of the nanostructure 450 include, but are not limited to nanotubes, nanowires, nanorods, nanoribbons, nanofilm, and nanoballs. In an embodiment, the nanostructure 450 is carbon based. A mass of olfactory receptor GPCRs 460 are deposited and bound to the nanostructure layer 450. The nanostructure layer 450 provides the electrical mechanism by which a pulse from activated analyte receptors, such as olfactory receptor GPCRs 460 is registered. For example, when specific analytes bind to analyte receptor molecules of the GPCRs 460, the equilibrium of the receptor molecules moves to an activated receptor state. The activated analyte receptor molecules modulate the contact resistance between a metal electrode of the source 440 and/or the drain 430 and the nanostructure layer 450, leading to a change in conductance. The change in conductance is registered and measured by electronic circuitry of the analyte-detection based electronic sensor device 400. When a specific analyte is in the vicinity of the analyte receptors of the GPCRs 460, a conductance modulation is generated by binding the analyte to the analyte receptor protein of the electronic sensor device 400. The analyte receptor protein is changed into an activated receptor state which causes a change in conductance. Detection of the analyte is achieved by measuring the change in conductance.

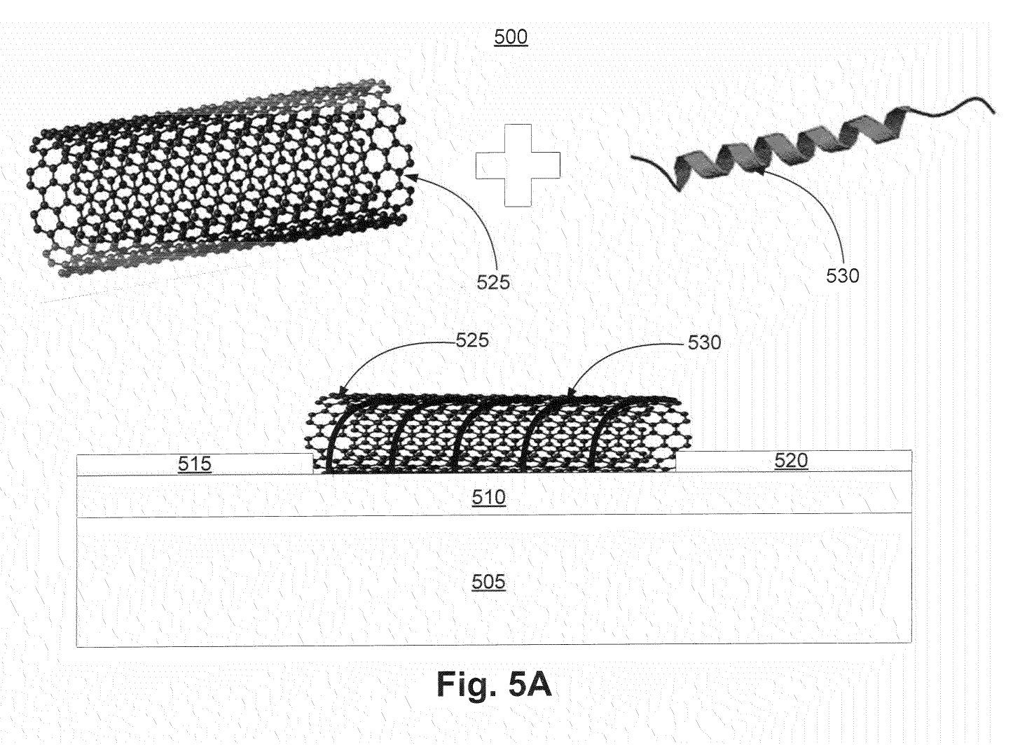

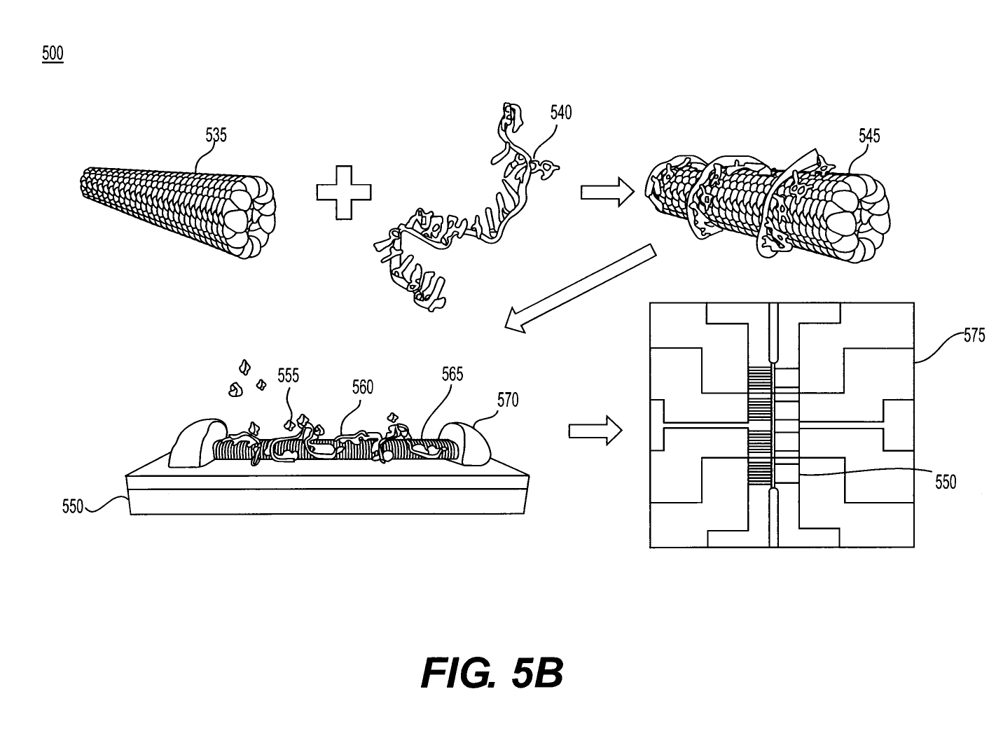

FIG. 5A illustrates an alternative exemplary detection-based electronic sensor device 500, such as an olfactory-based electronic device. Such a device may also be referred to as an artificial nose, an electronic nose, or an e-nose. A substrate 505, such as a silicon substrate has an oxide layer 510, such as silicon dioxide formed on the surface of the substrate 505, comprising the gate. A drain 515 is formed to one side of the substrate 505 on the oxide layer 510, and a source 520 is formed to another side of the substrate 505 on the oxide layer 510. A nanotube 525, such as a carbon nanotube, is wrapped with a single strand DNA (ss-DNA) 530 and is anchored to the surface of the oxide layer 510 (the gate) so that it is electrically connected to the source 520 and the drain 515. When a specific analyte is in the vicinity of the analyte receptors of the ss-DNA strand 530, a conductance modulation is generated by binding the odorant to the analyte receptor protein of the ss-DNA strand 530. The odorant interacts and binds transiently with the ss-DNA strand, modulating conduction in the gate 510 between the source 520 and the drain 515. Detection of the odorant is achieved by measuring the change in conductance.

FIG. 5B is a pictorial representation of an analyte-detection based electronic sensor device 500, such as an olfactory-based electronic device. A nanotube 535, such as a carbon nanotube, is wrapped with a DNA strand 540, such as a single-strand DNA (ss-DNA) or a double-strand DNA (ds-DNA) to produce a DNA-wrapped nanotube 545. The DNA-wrapped nanotube 545 is affixed on a semiconductor device 550. FIG. 5B illustrates analyte molecules 555, such as odorant molecules, some of which are bound to the DNA strand 560, which is wrapped about the carbon nanotube 565. An electrical conduit 570, such as a gold contact, affixes and electrically connects each end of the carbon nanotube 565 to the semiconductor device 550. The combined semiconductor device 550 and carbon nanotube 565 wrapped with the DNA strand 560 can be replicated and formed into an IC 575. The IC 575 shows four devices 550, but IC design techniques allow step-and-repeat fabrications of many such devices on one chip.

The e-nose outputs of a sensor can be compared to a local database (resident on a portable computing device or embedded within the sensor assembly itself) or a remote database of known odorants and other molecules, via the portable computer communications capability, either wireless or wired. The results of this analysis of the sensor readings can be displayed on a user interface of the portable computing device, and associated with the latent print from which it was generated.

A computing module can retrieve and apply identification models, which may be stored upon external resources, such as servers or databases in order to predict an identity, based upon sensor readout. Identification models are function approximations that map sensor readout information to identifications.

A prediction may require initial processing of the signal readout based upon identification model requirements. Processing may take place either on the computing module or on external resources. Processing requirements may include, but are not limited to signal filtering (highpass, lowpass, bandpass, bandstop, or notch filtering), denoising, time averaging, applying window functions, and numerical scaling of the signal readout data.

The identification model may be built or trained in numerous ways. A pattern-recognition or machine learning approach to identification model building uses an existing sensor readout database, where sensor readout data is paired with known identities. A model is built and optimized by minimizing the error between known mappings of sensor readout to identity and predicted mappings. Existing methods include, but are not limited to Bayes classification or regression, k-nearest-neighbor, ordinary/partial least squares classification or regression, support vector machines, decision trees, random forests, boosted trees, neural networks, and logistic regression. Error minimization may include application of signal processing techniques prior to modeling.

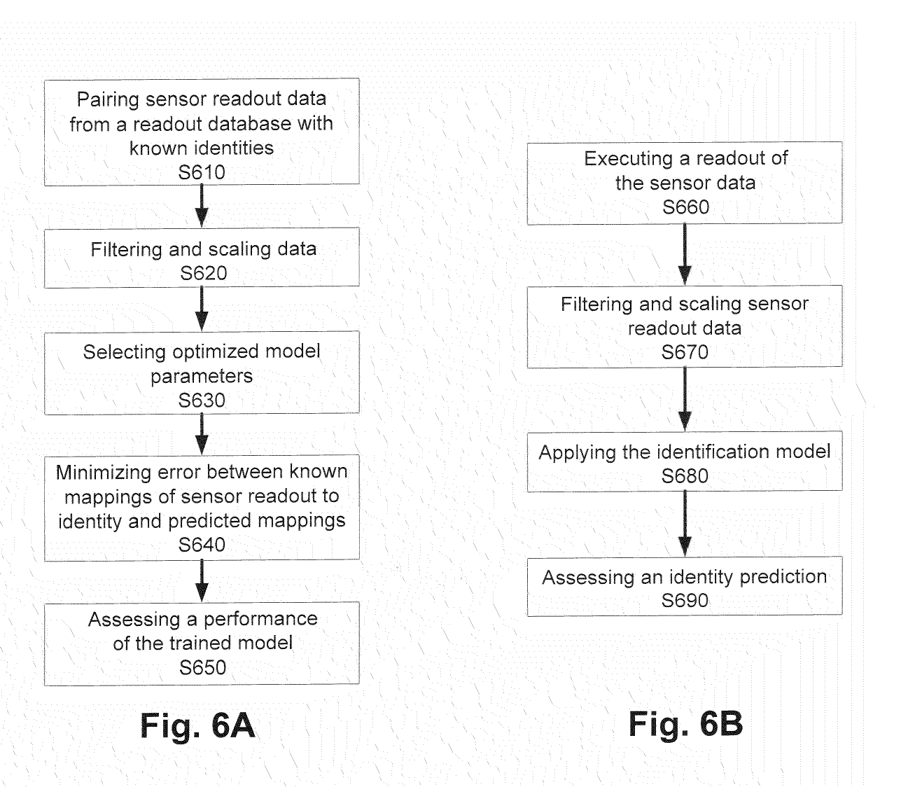

FIG. 6A is an exemplary algorithm for training a model. In step S610, sensor readout data from a readout database is paired with known identities. Training a model for optimization includes filtering and scaling data in step S620, as described above. Optimized model parameters are selected in step S630. A model is built by minimizing the error between known mappings of sensor readout to identity and predicted mappings in step S640. A performance of the trained model is assessed in step S650.

After the transformation of the sensor readout data, the computing module or external resource will apply the identification model, and a prediction of identity may be assessed. FIG. 6B is an exemplary algorithm for assessing the identification model. In step S660, a readout of the sensor data is executed. The sensor readout data is filtered and scaled in step S670. The identification model is applied in step S680 in which the format of the identity prediction will be specified in the requirements of the identification model. It may include true/false predictions, numerical predictions, confidence intervals, and uncertainty estimates. An identity prediction is assessed in step S690.

Alternative embodiments provide other approaches to identifying volatile chemicals emanating from a print in addition to a ss-DNA or ds-DNA e-nose, as described above. Alternative embodiments include, but are not limited to a combination of gas chromatography and mass spectroscopy, IR spectroscopy, UV-Vis spectroscopy, and nuclear magnetic resonance.

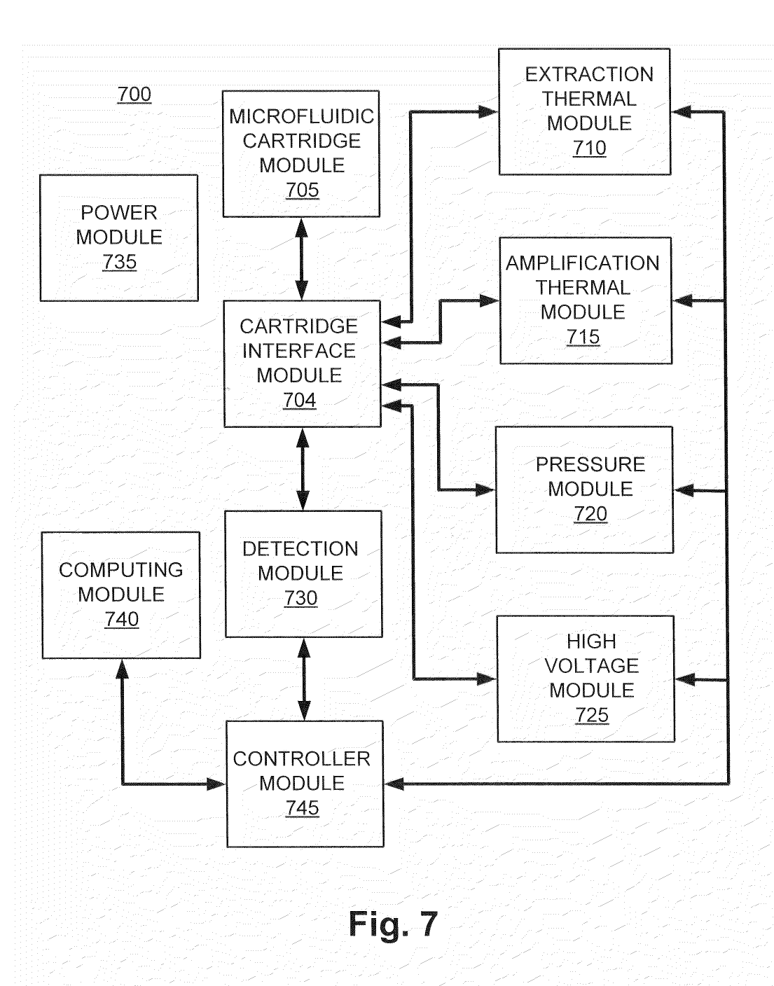

A third processing station includes a nucleic acid analyzer system, such as a system to determine the DNA content of a print swab. FIG. 7 shows a block diagram of an exemplary nucleic acid analyzer 700. As shown, the nucleic acid analyzer 700 can include a microfluidic cartridge module 705, a cartridge interface module 704, an extraction thermal module 710, an amplification thermal module 715, a pressure module 720, a high voltage module 725, a detection module 730, a power module 735, a computing module 740, and a controller module 745. The modules can be operably connected as shown in FIG. 7. In embodiments, the modules can also be combined or more than one of each module may be present in a nucleic acid analyzer.

The nucleic acid analyzer 700 is capable of performing nucleic acid analysis using a microfluidic cartridge. The nucleic acid analyzer 700 is designed to use liquid volumes on the order of micro-liters or less. By using micro-liter liquid volumes, nucleic analysis can be performed in reduced time as compared to nucleic acid analysis using larger volumes.

The microfluidic cartridge module 705 is configured to accept one or more microfluidic samples (not shown). The cartridge interface module 704 is configured to operably couple the microfluidic cartridge module 705 to the other modules. In an embodiment, some of the other modules, such as the detection module 730, the extraction thermal module 710, the amplification thermal module 715, and the like, can be integrated in the cartridge interface module 704. The microfluidic cartridge can include a micro-to-macro interface and features that allow the microfluidic cartridge to be acted upon by other components of the nucleic acid analyzer 700. The microfluidic cartridge can be a disposable cartridge, such as a single-use cartridge. In general, microfluidic cartridges can include various features for performing any of nucleic acid extraction, nucleic acid amplification, and nucleic acid separation. Defined within the microfluidic cartridge is a fluidic network formed from fluidic channels, fluidic chambers and/or reservoirs, and other features for performing nucleic acid extraction, nucleic acid amplification, and/or nucleic acid separation. The microfluidic cartridge can be constructed from any suitable material. As examples, the microfluidic cartridge can be constructed from a plastic, polymeric material, glass, and the like. Additionally, the microfluidic cartridge can be constructed from multiple types of materials.

The extraction thermal module 710 is configured to impart suitable temperatures for nucleic acid extraction. The extraction thermal module 710 can be controlled by the controller module 745. The extraction thermal module 710 can be coupled to a cartridge or a sample acceptor during nucleic acid extraction. The extraction thermal module 710 can perform contact and/or non-contact thermal heating. In an example, the extraction thermal module 710 includes one or more contact heating units. Heating with the extraction thermal module 710 can facilitate the extraction of nucleic acids with thermophilic enzymes.

The amplification thermal module 715 is configured to impart suitable temperatures to the microfluidic cartridge during nucleic acid amplification. The amplification thermal module 715 can be controlled by the controller module 745. In embodiments, the amplification thermal module 715 can be configured to impart thermal gradients and perform temperature sensing in a thermal cycling process in an amplification chamber of the microfluidic cartridge. The amplification thermal module 715 can perform contact and/or non-contact thermal heating. In an example, the amplification thermal module 715 includes a non-contact heating unit, such as an infrared light source. Further, the amplification thermal module 715 can include a temperature sensing unit. In an embodiment, the temperature sensing unit is an infrared pyrometer that measures blackbody radiation to determine the temperature of a selected portion of the microfluidic cartridge. Further, in embodiments, a single thermal module can be configured to impart temperature changes for both extraction and amplification, as necessary, using the same heating means.

The pressure module 720 is operably coupled to the microfluidic cartridge by, for example, the micro-to-macro interface. The pressure module 720 can be controlled by the controller module 745. The pressure module 720 is configured to provide pressures and/or vacuums (i.e., positive and/or negative pressures) to the microfluidic cartridge to move fluid within a fluidic network of the microfluidic cartridge. In other words, the pressure module 720 can effectuate hydrodynamic movement using, for example, pneumatic pressure in the microfluidic cartridge. In an embodiment, the pressure module 720 is coupled to one or more clusters of vent ports on the microfluidic cartridge at the micro-to-macro interface. The pressure module 720 can connect a solenoid manifold to the plurality of vent ports of the microfluidic cartridge at the micro-to-macro interface. The pressure module 720 can impart pressure to each vent port independently to move fluid through the fluidic network in the microfluidic cartridge. In an embodiment, the microfluidic cartridge has one or more valves that are configured to be actuated by the pressure module 720. The pressure module 720 can include a pressure/vacuum system, such as a pneumatic pressure/vacuum system, to suitably control hydrodynamic movement in the fluidic network of the microfluidic cartridge.

The power module 735 generates various operation powers for various components of the nucleic acid analyzer 700. In an example, the nucleic acid analyzer 700 is implemented using a modular design. Each module of the nucleic acid analyzer 700 requires an operation power supply, which can be different from the other modules. The power module 735 can receive an AC power input, such as 100-240 V, 50-60 Hz, single phase AC power from a power outlet. The power module 735 can use the AC power input to generate 5 V, 12 V, 24 V, and the like, to provide operation powers for the various components of the nucleic acid analyzer 700. In other embodiments, the power module 735 can be a battery.

The power module 735 also imparts power to the high voltage module 725 as required for nucleic acid processes on the microfluidic cartridge, such as electrophoretic separation. The power module 735 can implement various protective functions, such as power outage protection, graceful shut-down, and the like, to protect the various components and data against power failure. In an embodiment, the power module 735 includes a back-up power, such as a battery module, to support one or more protective functions, such as graceful shut-down.

The high voltage module 725 receives power from the power module 735 and generates high voltages such as 1000 V, 2000 V, and the like, required for nucleic acid processes on the microfluidic cartridge, such as electrophoretic separation. The high voltage module 725 can apply the high voltages to the microfluidic cartridge under control of the controller module 745. For example, the high voltage module 725 includes an interface that applies the high voltages to electrodes on the microfluidic cartridge to induce electro-kinetic injection and/or electrophoretic separation.

The detection module 730 includes components configured to detect labeled or dyed nucleic acids. The detection module 730 can be controlled by the controller module 745. In an embodiment, the detection module 730 is configured for fluorescence detection, such as multicolor fluorescence detection. The detection module 730 can include a laser source unit, an optical unit, and a detector unit. The optical unit includes a set of optics. In an embodiment, the optical unit includes a self-calibrating array of confocal optical components. The laser source unit emits a laser beam. In an example, the laser source unit includes an argon-ion laser unit. In another example, the laser source unit includes a solid state laser, such as a coherent sapphire optically pumped semiconductor laser unit. The solid state laser has the advantages of reduced size, weight and power consumption.

In operation, the set of optics can direct the laser beam to penetrate a detection region of a separation channel in the microfluidic cartridge. The laser beam can excite fluorescent molecules attached to nucleic acids to emit fluorescence. The set of optics can then collect and direct the emitted fluorescence to the detector unit for detection. The detector unit can convert the detected fluorescence into data for subsequent processing by the computing module 740. An exemplary detection technique is disclosed by co-pending U.S. application Ser. No. 13/273,947 entitled, “Micro Fluidic Optic Design,” which is hereby incorporated herein by reference in its entirety.

The computing module 740 includes computing and communication units. The computing module 740 is operably coupled to the controller module 745. The computing module 740 can provide a user interface. The user interface can provide the status of the nucleic acid analyzer 700 and can receive user instructions for controlling the operation of the nucleic acid analyzer 700. The computing module 740 includes various storage media to store software instructions and data. The computing module 740 can include nucleic analysis software that can perform data processing based on raw data obtained from the detection module 730. In addition, the computing module 740 can be coupled to external processing units, such as a database, a server, and the like to further process the data obtained from nucleic acid analysis.

The touch DNA analysis provided by a rapid DNA analysis device can be compared locally to a database of suspected individuals on a portable computing device, or it can be sent to a local DNA index system (LDIS), a state DNA index system (SDIS), or a national DNA index system (NDIS) for remote comparison. The results of the analysis of the rapid DNA sensor output can be displayed on a user interface of the portable computing device and associated with the latent print from which it was generated.

The controller module 745 can receive status signals and feedback signals from the various components and provide control signals to the various components according to a nucleic acid analysis procedure. In addition, the controller module 745 can provide the status signals to the computing module 740 to inform a user of the status of nucleic acid analysis. Further, the controller module 745 can receive user instructions from the computing module 740 and can provide control signals to the various components based on user instructions.

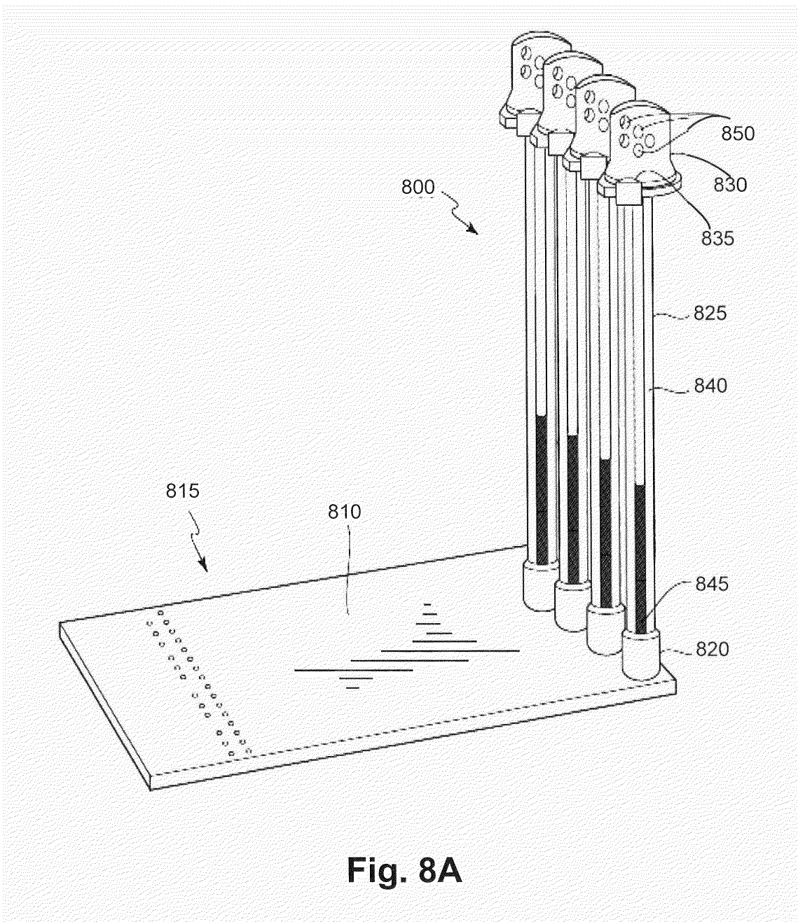

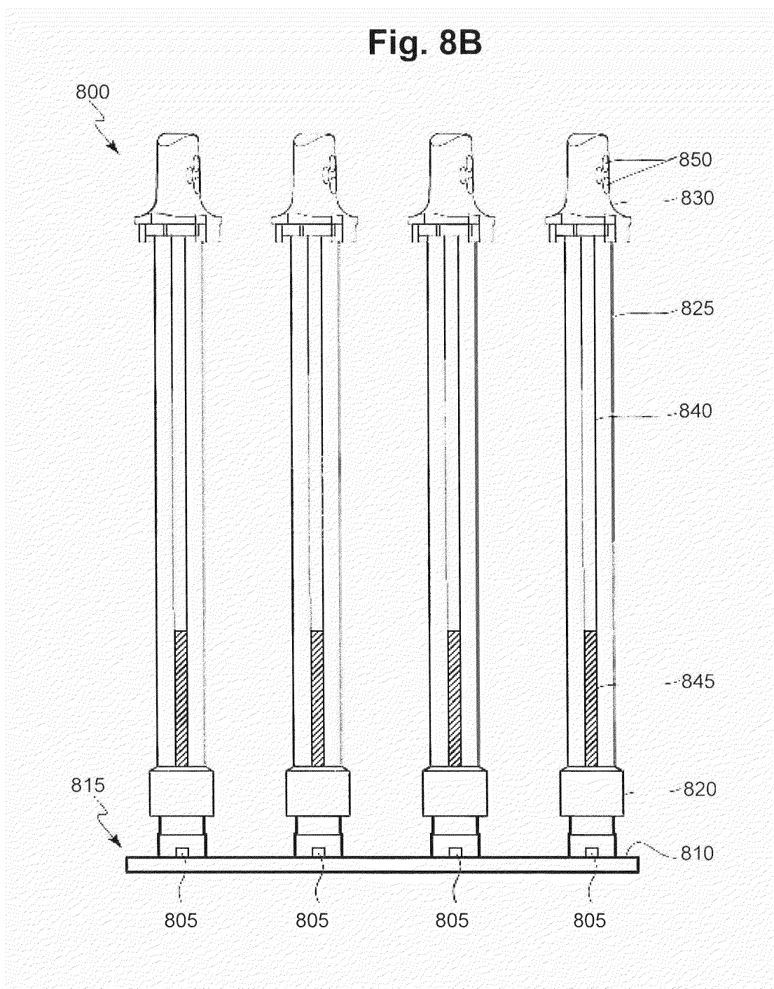

FIGS. 8A and 8B illustrate an exemplary embodiment of a microfluidic cartridge 815 having a plurality of sample acceptors 800. The sample acceptors 800 are fluidically coupled to a plurality of sample inputs 805 formed on an outer surface 810 of the microfluidic cartridge 815. As shown, each sample input 805 includes a portion surrounding an opening that protrudes from the outer surface 810 of the microfluidic cartridge 815. In FIGS. 8A and 8B, four sample acceptors 800 are fluidically coupled to four sample inputs 805 of the microfluidic cartridge 815. In other embodiments, the microfluidic cartridge 815 can include less than four sample inputs 805, including a single sample input 805, or more than four sample inputs 805 for fluidically coupling the same number of sample acceptors 800. The sample inputs 805, as well as the sample acceptors 800, can be of the same or different types. As shown, the sample acceptors 800 and the sample inputs 805 are of the same type. Alternatively, one or more of the sample acceptors 800 and the sample inputs 805 can be of different types.

As further shown, each sample acceptor 800 includes an input-connectable portion 820, an acceptor portion 825, and a detachable portion 830 for sample collection. The input-connectable portion 820 is at one end of the acceptor portion 825. The acceptor portion 825 is in the form of a barrel similar to a syringe barrel. The input-matable portion 820 can be configured to be coupled to the sample input 805 to form a fluid-tight seal. The input-matable portion 820 and the sample input 805 can be based on any small-scale fluid-fitting system. In embodiments, the input-matable portion 820 and the sample input 805 each have a universal connector selected from the group consisting of Luer-Lok connectors, threaded connectors, and flanged connectors. For example, the input-matable portion 820 and the sample input 805 can be based on a Luer-Lok fitting system. In an embodiment, the sample input 805 is threaded such as to be a female Luer-Lok fitting and the input-matable portion 820 is based on a complementary male Luer-Lok fitting that has an inner flange configured to fit inside the opening of the sample input 805 and a second outer flange that is threaded and configured to be screw-fitted onto the threaded sample input 805.

The detachable portion 830 is configured to be removed from the acceptor portion 825 to collect a biological sample and again coupled to the acceptor portion 825 after collection of the biological sample has been completed. To effectuate removable coupling, the detachable portion 830 includes a flanged grip 835. The flanged grip 835 can be configured to be reversibly coupled to a complementary end of the acceptor portion 825. Extending from the flanged grip 835 is an elongated member 840 that includes a sample collection portion 845. The sample collection portion 845 can be in the form of a swab.

Nucleic acid extraction can be performed when the microfluidic cartridge 815 is coupled to a pressure module of a nucleic acid analyzer. The pressure module can provide positive and/or negative pressure to force an enzymatic mixture from an extraction mixture reservoir of the microfluidic cartridge 815 into the sample acceptor 800 in order to perform nucleic acid extraction on a biological sample presented by the sample acceptor 800. To aid enzymatic digestion, the pressure module, through positive and/or negative pressure, can move the enzymatic mixture in a back-and-forth motion within the sample acceptor 800 and the extraction mixture reservoir of the microfluidic cartridge 815. The flanged grip 835 of the sample acceptor 800 can be gas permeable to permit gas (e.g., air) to exit the sample acceptor 800. As shown, the sample acceptor 800 is made gas permeable by including openings 850 defined in the flanged grip 835.

The microfluidic cartridge 815 can include a vent port in fluid communication with the extraction mixture reservoir, which can place the pressure module in serial fluid communication with the sample acceptor 800 through the extraction mixture reservoir and the sample input 805. In embodiments, the pressure module applies positive and/or negative pressure to the distal end of the extraction mixture reservoir to force a volume of the enzymatic mixture through the sample input 805 into the sample acceptor 800, where the enzymatic mixture can submerge the biological sample presented on the sample collection portion 845 of the sample acceptor 800. The pressure module, under control of a controller module, can then force the enzymatic mixture and dissolved biological sample back into the extraction mixture reservoir. The pressure module can revert at least a major portion of the enzymatic/biological sample mixture back into the sample acceptor 800. This back-and-forth motion can be continued by operation of the pressure module using positive and/or negative pressure, such as pneumatic pressure, and discontinued once nucleic acid extraction is completed. The turbidity associated with the back-and-forth motion can aid nucleic acid extraction and can produce a well-mixed solution of extracted nucleic acids.

During nucleic acid extraction, the sample acceptor 800 can be coupled to an extraction thermal module of a nucleic acid analyzer. As discussed above, the extraction thermal module can heat the enzymatic mixture to promote enzymatic digestion of cellular components (other than nucleic acids) of the biological sample presented by the sample acceptor 800.

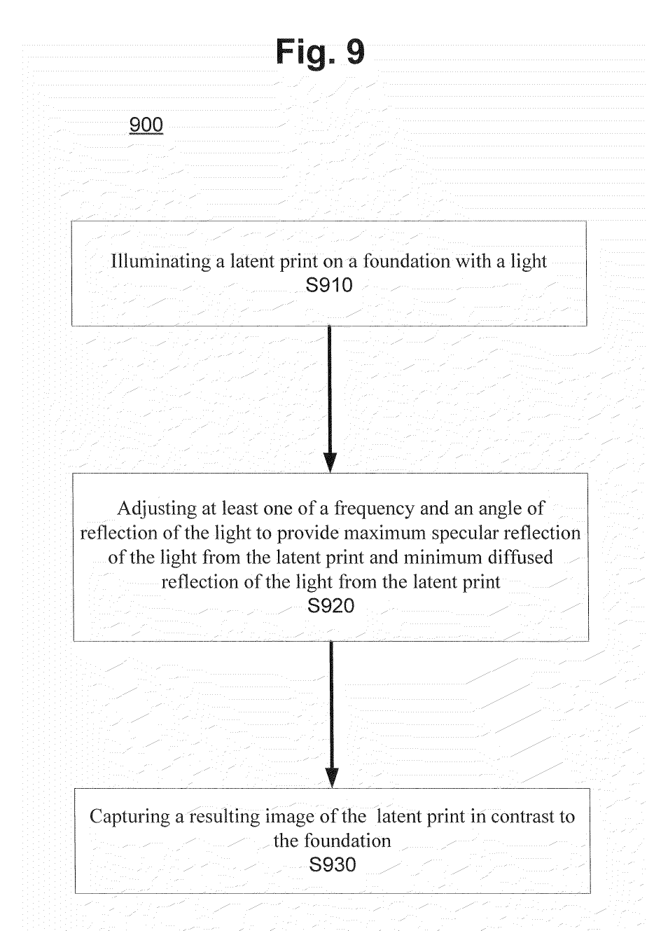

FIG. 9 is a flowchart of an exemplary method 900 of capturing a print, such as a fingerprint or a palm print. A latent print is illuminated on a foundation with a light in step S910. At least one of a frequency and an angle of reflection of the light is adjusted in step S920 to provide maximum specular reflection of the light from the latent print and to provide minimum diffused reflection of the light from the latent print. A resulting image of the latent print in contrast to the foundation is captured in step S930. In an embodiment, the latent print can be an organic-based latent print.

Method 900 can also include adjusting the angle of reflection to be nearly equal to an angle of incidence relative to a surface of the sample to achieve the maximum specular reflection of the light. The adjusting can further include aligning a light source and a light detector at a critical alignment angle to create and capture the specular reflection from an illuminated surface of the sample. The adjusting can further include setting a numerical aperture of an optical system coupled to a light detector to zero. The adjusting can also include setting numerical apertures of an optical system and the light source to be substantially equal and opposite.

Method 900 can also include adjusting a wavelength or wavelength range of the light according to a material or surface texture of the foundation. The adjusting can further include adjusting one or more filters, activating or de-activating one or more filters, separating out specific wavelengths using refraction or reflection techniques, and activating one or more individual light sources configured to produce a desired wavelength.

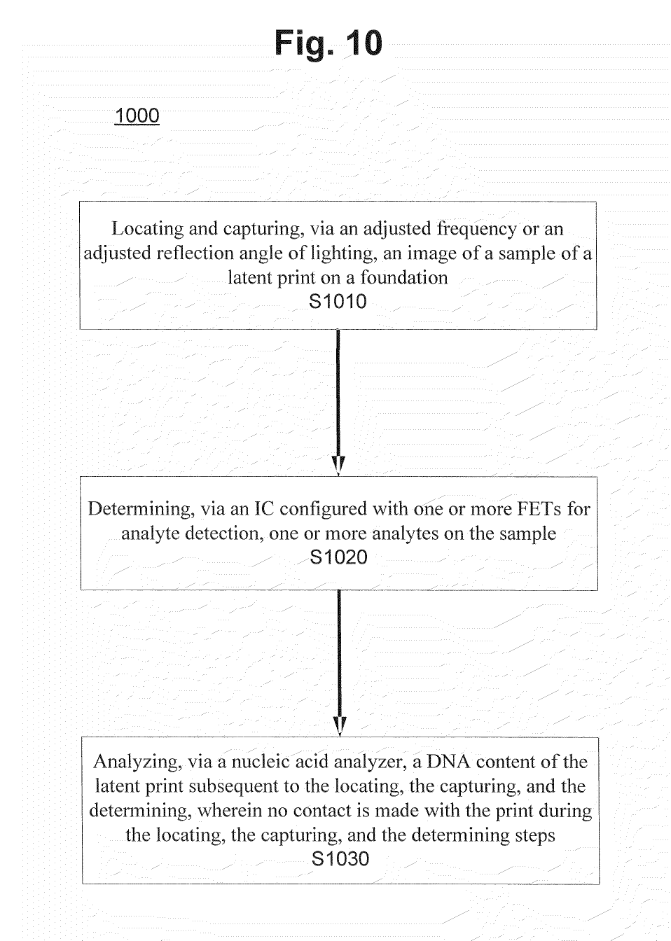

FIG. 10 is a flowchart of an exemplary method 1000 of identifying a print, such as a fingerprint or a palm print. An image of a sample of a latent print is located and captured on a foundation, via an adjusted frequency or an adjusted reflection angle of lighting in step S1010. One or more analytes on the sample are determined, via an IC configured with one or more FETs for analyte detection in step S1020. A DNA content of the latent print is analyzed, via a nucleic acid analyzer, subsequent to the locating, the capturing, and the determining in step S1030. No contact is made with the print during the locating, the capturing, and the determining steps. In an embodiment, the latent print can be an organic-based latent print. In another embodiment, the one or more analytes can be one or more organic-based analytes.

Method 1000 can also include maximizing specular reflection of the lighting from the latent print and minimizing diffused reflection of the lighting from the foundation by adjusting an angle of reflection of the lighting to be nearly equal to an angle of incidence of the lighting relative to a surface of the sample. Method 1000 can also include adjusting a wavelength or wavelength range of the lighting according to a material or surface texture of the foundation.

Method 1000 can also include activating a single-strand DNA (ss-DNA) strand bound to a nanotube by an analyte interacting with the ss-DNA strand. The nanotube comprises an active component of an FET gate and is electrically coupled to a source and a drain of the IC and is configured to measure a change in conductance upon the activating. Method 1000 can also include activating a mass of GPCRs bound to a nanostructure layer of an FET gate of one of the FETs. The nanostructure layer is electrically coupled to a source and a drain of the FET and is configured to measure a change in conductance when the GPCRs are activated by an analyte specific to the GPCRs.

Method 1000 can also include extracting, amplifying, separating, and identifying the DNA content of the latent print via a microfluidic cartridge of the nucleic acid analyzer. The IC can be configured with a FET functionalized with olfactory receptors, or other types of functionalization agents selected for analyte detection. The one or more FETs can include one or more ChemFETs or one or more BioFETs.

Embodiments herein describe a system of identifying a print, which includes an image-capturing and lighting optical system configured to maximize specular reflection of light reflected from a print and to minimize diffused reflection of light reflected from a background surface of the print via adjustment of at least one of a frequency and a reflection angle of the light emitted upon a sample of the print. The system also includes an IC having one or more FETs with a nanostructure configured to detect a plurality of analytes from the print. The system also includes a nucleic acid analyzer configured to process the print and to determine a DNA content of the print. The nucleic acid analyzer can also include a microfluidic cartridge configured to extract, amplify, and separate a DNA content of the print and to identify the DNA content of the print. No contact is made with the print, while being subjected to processing by the image-capturing and lighting optical system and the IC. In an embodiment, the print can be an organic-based print. In another embodiment, the plurality of analytes can be a plurality of organic-based analytes.

The image-capturing and lighting optical system can also include an angle of reflection nearly equal to an angle of incidence of the emitted light relative to a surface of the sample, and configured to achieve a maximum specular reflection of the emitted light from the print and a minimum diffused reflection of the emitted light from the background surface of the print. The image-capturing and lighting optical system can also include one or more filters configured to adjust a wavelength of the emitted light according to a material or surface texture of the background surface of the print.

The IC can also include a mass of GPCRs bound to a nanostructured surface including a gate of one of the FETs. The nanostructure is electrically coupled to a source and a drain of the FET and is configured to measure a change in conductance when the GPCRs are activated by an analyte specific to the GPCRs. The IC can also include a DNA strand bound to a nanotube including a gate of one of the FETs. The nanotube is electrically coupled to a source and a drain of the FET and is configured to measure a change in conductance when the DNA strand is activated by an analyte interacting with the DNA strand. The nucleic acid analyzer can also include a microfluidic cartridge configured to extract, amplify, and separate a DNA content of the print and to identify the DNA content of the print.

Embodiments disclosed herein can incorporate a laptop, pad computer, palmtop, smartphone, or other portable computing devices with corresponding wireless or wired input and output communications capabilities. The portable computer collects latent print images of a latent print imager, associated e-nose outputs, and associated touch DNA outputs of a rapid DNA device. The computer associates the disparate sensor outputs for the purposes of chain of evidence, and can provide further computing capability if it isn't included in the sensor system itself.

The portable computing device can process outputs from any of the three sensors individually, or associate them in any combination that correlates to the nature of the evidence presented. Records of such outputs, their associations and meta-data can be stored locally and/or transmitted to a suitable central repository for further evidence processing and potential further investigatory or judicial use.

Systems and methods of embodiments described herein provide the advantage of having multiple avenues to locate, collect, and identify information retrieved from a print, without adulterating or disturbing the print until it is swabbed for touch DNA. Latent image identification and analyte-based identification of the print provide valuable information without disturbing it. In addition, the print can ultimately be processed for possible touch DNA identification. A maximum number of unadulterated skin cells are provided for DNA identification using embodiments described herein. Also, if a print is smudged or for any other reason does not have adequate detail for latent imaging, the e-nose analysis and DNA sequencing can still be used towards identifying the print, or associating it with other un-smudged prints, rendering useful evidence which might otherwise be ignored or discarded.

While the invention has been described in conjunction with the specific exemplary embodiments thereof, it is evident that many alternatives, modifications, and variations will be apparent to those skilled in the art. Accordingly, exemplary embodiments as set forth herein are intended to be illustrative, not limiting. There are changes that can be made without departing from the spirit and scope of the invention.

1. A method of capturing a print, the method comprising:
illuminating a latent print on a foundation with a light;
adjusting at least one of a frequency and an angle of reflection of the light to provide maximum specular reflection of the light from the latent print and minimum diffused reflection of the light from the latent print; and
capturing a resulting image of the latent print in contrast to the foundation.

2. The method of claim 1, further comprising:
adjusting the angle of reflection to be nearly equal to an angle of incidence relative to a surface of the latent print to achieve the maximum specular reflection of the light.

3. The method of claim 2, wherein the adjusting further comprises:
aligning a light source and a light detector at a critical alignment angle to create and capture the specular reflection from an illuminated surface of the latent print.

4. The method of claim 2, wherein the adjusting further comprises:
setting a numerical aperture of an optical system coupled to a light detector to zero.

5. The method of claim 2, wherein the adjusting further comprises:
setting numerical apertures of an optical system and the light source to be substantially equal and opposite.

6. The method of claim 1, further comprising:
adjusting a wavelength or wavelength range of the light according to a material or surface texture of the foundation.

7. The method of claim 6, wherein the adjusting further comprises:
adjusting one or more filters, activating or de-activating one or more filters, separating out specific wavelengths using refraction or reflection techniques, and activating one or more individual light sources configured to produce a desired wavelength.

8. The method of claim 1, wherein the latent print includes an organic-based latent print.

9. A method of identifying a print, the method comprising:
locating and capturing, via an adjusted frequency or an adjusted reflection angle of lighting, an image of a sample of a latent print on a foundation;
determining, via an integrated circuit (IC) configured with one or more Field Effect Transistors (FETs) for analyte detection, one or more analytes on the sample; and
analyzing, via a nucleic acid analyzer, a DNA content of the latent print subsequent to the locating, the capturing, and the determining, wherein no contact is made with the print during the locating, the capturing, and the determining steps.

10. The method of claim 9, wherein the capturing further comprises:
maximizing specular reflection of the lighting from the latent print and minimizing diffused reflection of the lighting from the foundation by adjusting an angle of reflection of the lighting to be nearly equal to an angle of incidence of the lighting relative to a surface of the sample.

11. The method of claim 9, wherein the capturing further comprises:
adjusting a wavelength or wavelength range of the lighting according to a material or surface texture of the foundation.

12. The method of claim 9, wherein the determining further comprises:
activating a single-strand DNA (ss-DNA) strand bound to a nanotube by an analyte interacting with the ss-DNA strand, wherein the nanotube comprises an active component of an FET gate and is electrically coupled to a source and a drain of the IC and is configured to measure a change in conductance upon the activating.

13. The method of claim 9, wherein the determining further comprises:
activating a mass of G protein-coupled receptors (GPCRs) bound to a nanostructure layer of an FET gate of one of the FETs, wherein the nanostructure layer is electrically coupled to a source and a drain of the FET and is configured to measure a change in conductance when the GPCRs are activated by an analyte specific to the GPCRs.

14. The method of claim 9, further comprising:
extracting, amplifying, separating, and identifying the DNA content of the latent print via a microfluidic cartridge of the nucleic acid analyzer.

15. The method of claim 9, wherein the IC is configured with a FET functionalized with olfactory receptors.

16. The method of claim 9, wherein the one or more FETs include one or more chemically-based FETs (ChemFETs) or one or more biologically-based FETs (BioFETs).

17. The method of claim 9, wherein the latent print includes an organic-based latent print and the one or more analytes include one or more organic-based analytes.

18. A system of identifying a print, the system comprising:
an image-capturing and lighting optical system configured to maximize specular reflection of light reflected from a print and to minimize diffused reflection of light reflected from a background surface of the print via adjustment of at least one of a frequency and a reflection angle of the light emitted upon a sample of the print;
an integrated circuit (IC) having one or more Field Effect Transistors (FETs) with a nanostructure configured to detect a plurality of analytes from the print; and
a nucleic acid analyzer configured to process the print and to determine a DNA content of the print, wherein no contact is made with the print, while being subjected to processing by the image-capturing and lighting optical system and the IC.

19. The system of claim 18, wherein the image-capturing and lighting optical system further comprises:
an angle of reflection that is nearly equal to an angle of incidence of the emitted light relative to a surface of the sample and configured to achieve a maximum specular reflection of the emitted light from the print and a minimum diffused reflection of the emitted light from the background surface of the print; and
one or more filters configured to adjust a wavelength of the emitted light according to a material or surface texture of the background surface of the print.

20. The system of claim 18, wherein the IC further comprises:
a mass of G protein-coupled receptors (GPCRs) bound to a nanostructured surface including a gate of one of the FETs, wherein the nanostructure is electrically coupled to a source and a drain of the FET and is configured to measure a change in conductance when the GPCRs are activated by an analyte specific to the GPCRs.

21. The system of claim 18, wherein the IC further comprises:
a DNA strand bound to a nanotube including a gate of one of the FETs, wherein the nanotube is electrically coupled to a source and a drain of the FET and is configured to measure a change in conductance when the DNA strand is activated by an analyte interacting with the DNA strand.

22. The system of claim 18, wherein the nucleic acid analyzer further comprises:
a microfluidic cartridge configured to extract, amplify, and separate a DNA content of the print and to identify the DNA content of the print.

23. The system of claim 18, wherein the print includes an organic-based print and the plurality of analytes includes a plurality of organic-based analytes.
